(19) United States
(12) Patent Application Publication
(10) Pub. No.: US 2004/0020494 A1
(43) Pub. Date: Feb. 5, 2004

(54) Contraceptive diaphragm with a superelastic member

(76) Inventor: Janet Walsh Burpee, Fair Haven, NJ (US)

Correspondence Address: Janet W. Burpee, 56 Buttonwood Drive, Fair Haven, NJ, 07704, US

(21) Appl. No.: 10/214,950

(22) Filed: Jul. 31, 2002

(51) Int. Cl. A61F 6/06

(52) U.S. Cl. 128/837

(57) ABSTRACT

This contraceptive diaphragm is made with a ring or member made at least in part from a shape memory alloy. The shape memory alloy is superelastic at body temperature. When deployed this superelastic ring or member applies a declining but more constant force on the vaginal wall than the rings used in current contraceptive diaphragms. This more constant force enables a tighter fit in the event that the woman using the diaphragm was sized incorrectly. It also allows the physician a greater margin of error when sizing a woman for a diaphragm. Because the superelastic ring should provide a more consistent, snug fit than existing diaphragms, it should more reliably prevent the passage of sperm and seminal fluid.

### BACKGROUND OF THE INVENTION

Contraceptive diaphragms are well-known medical devices, deployed as a barrier in the vagina, typically to prevent the passage of sperm into the vagina. Contraceptive diaphragms are one of many contraceptive barrier devices and have been around since the early 1900's. Most current contraceptive diaphragms have an embedded ring composed of a polymer or steel. This ring typically forms the diaphragm's outer rim that encircles and is embedded within a thin polymer sheet. The polymer sheet forms a dome-like shape, and the ring holds the dome-like shape around it's perimeter. The ring can be compressed enabling the device to become smaller and allowing for easy insertion into the vagina. Once in the vagina, the ring is allowed to open again under its own forces. When the ring of the diaphragm opens, it forms a seal along the vaginal wall. The polymer layer of the diaphragm surrounding the ring makes contact with the vaginal wall. This contact prevents sperm and seminal fluid from passing into the uterus and other parts of the female anatomy. Current devices are placed into the vagina by folding or collapsing the device, inserting the device into the vagina at a proper angle, and allowing the device to open under its own unloading forces. Currently, the mechanical properties of the rings in many existing diaphragms cause them to open with a force that rapidly decreases as the diaphragm opens.

### SUMMARY OF THE INVENTION

The disclosed invention is a contraceptive diaphragm which fits, at least in part, inside the vagina. This diaphragm has a ring or member which is made at least in part from a shape memory alloy. The shape memory alloy, which is superelastic at body temperature, enables the diaphragm to apply a declining but more uniform expansion force than current diaphragms throughout most of its unfolding within the vagina. That is, when the diaphragm begins to unfold from the compressed configuration, the force exerted by the diaphragm against the vaginal wall does not rapidly decline throughout most of its opening. Unlike current devices, in this device the rapid decline in forces occurs after the unfolding of the diaphragm has neared completion. A more uniform opening force offers three significant benefits. First, the more uniform force increases the likelihood that the device continues to open after initial insertion. In other words, the mechanical properties of the materials used in the ring increases the likelihood that the forces unfolding the diaphragm exceed the forces exerted on the device by the vaginal wall. Second, if the diaphragm is misplaced and the forces on the diaphragm do not allow it to open, the higher opening force may still enable a seal to form between the vagina and the diaphragm. Third, the more uniform opening force ensures a more secure fit if the device is too large for the patient. A physician typically fits a patient with a set diaphragm size. If the diaphragm size is off slightly, or the woman's anatomy changes slightly over time, a diaphragm with a superelastic member would continue to apply a force during unfolding which could help to maintain a seal between itself and the vaginal wall.

Other barrier contraceptive methods—such as a cervical cap or a female condom—which are inserted at least in part into the vagina could also use a shape memory ring or member to provide a more uniform opening force.

### BRIEF DESCRIPTION OF THE DRAWINGS

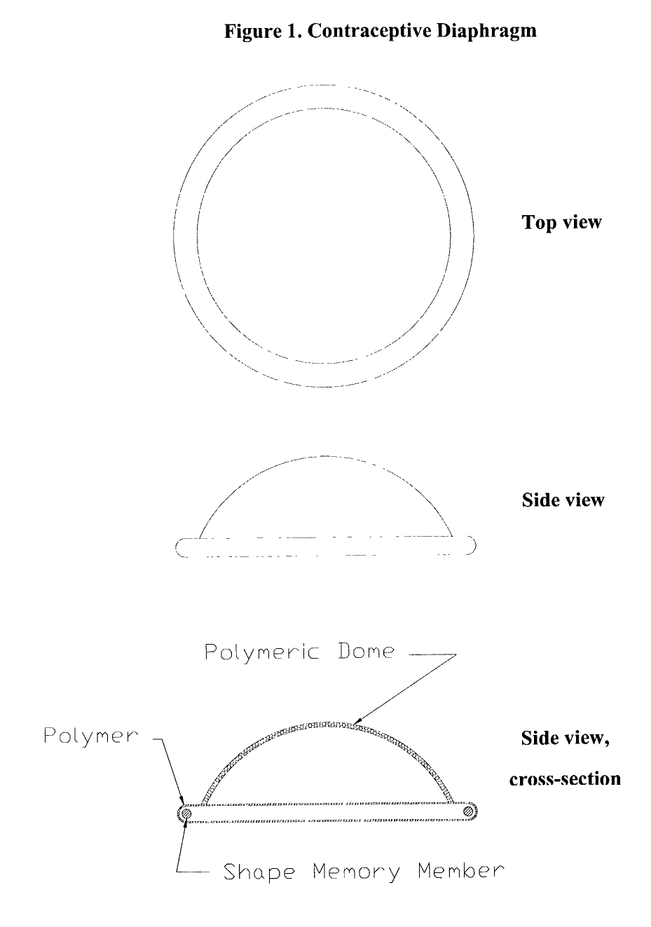

1. FIG. 1 depicts the general shape and cut-away of the proposed diaphragm. The material surrounding the metal ring is a thin layer of a polymer such as rubber (typically silicone and latex), or polyurethane. The inner metal ring is made of a shape memory alloy such as nickel-titanium.

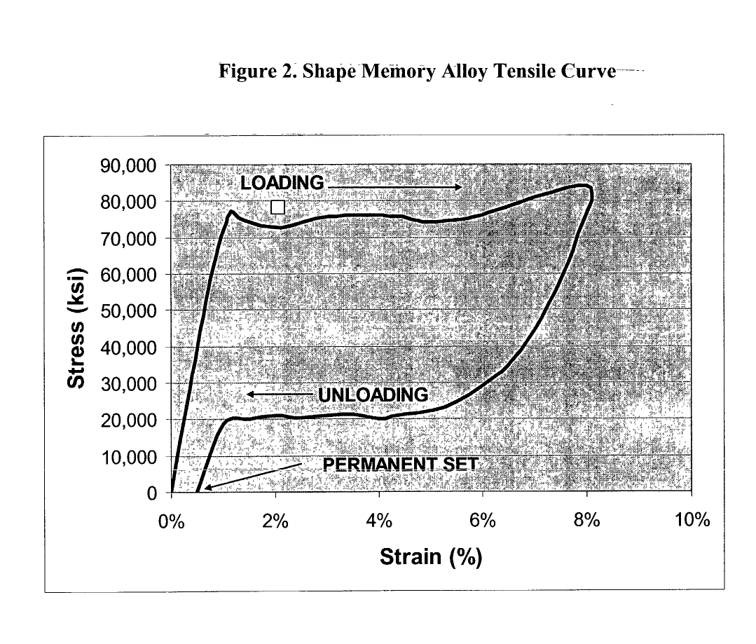

2. FIG. 2 depicts a typical tensile curve at body temperature for a superelastic shape memory alloy (example shown is a nickel-titanium alloy) showing the mechanical loading and unloading behavior. This curve shows loading to 8% followed by unloading. The unloading of the tensile curve correlates to the unfolding of the superelastic ring of the device.

### DESCRIPTION OF THE CLAIMS

What is claimed is:

1. A contraceptive device in the form of a dome shaped diaphragm with a ring or a ring-like member embedded or partially embedded along the perimeter of the polymer dome material where the ring is
(a) superelastic at room temperature and body temperature
(b) constructed of a material which is selected from the group of:
i. any alloy or group of alloys which demonstrates the shape memory behavior; OR
ii. such alloy(s) as described in (i) in combination with (including entwined or side-by-side) another material such as a polymer or metal; OR
iii. alloys comprised of 55.4% to 56.1% nickel and 43.0% to 44.6% titanium; OR
iv. such alloys as described in (iii) in combination with (including entwined or side-by-side) another material such as a polymer or metal; OR
v. alloys comprised of 44.5% to 47% titanium, with 0.1 to 2.5% of one or more of the elements including tantalum, iron, molybdenum, manganese, cobalt, chromium, vanadium, niobium, hafnium, zirconium, and tungsten, and the balance nickel; OR
vi. such alloys as described in (v) in combination with (including entwined or side-by-side) another material such as a polymer or metal.
(c) configured at least in part as:
i. a single shape memory wire, tube, filled-tube, or strip, or
ii. a braided or otherwise twisted shape memory wire, tube, filled-tube, and/or strip

2. A device according to claim 1 where the superelastic ring or ring-like member is superelastic at body temperature, but shape memory at room temperature.

3. A contraceptive device in the form of a cervical cap with a ring or ring-like member embedded or partially embedded along the perimeter of the polymer material where the ring is
(a) superelastic at room temperature and body temperature
(b) constructed of a material which is selected from the group of:
i. any alloy or group of alloys which demonstrates the shape memory behavior; OR
ii. such alloy(s) as described in (i) in combination with (including entwined or side-by-side) another material such as a polymer or metal; OR
iii. alloys comprised of 55.4% to 56.1% nickel and 43.9% to 44.6% titanium; OR
iv. such alloys as described in (iii) in combination with (including entwined or side-by-side) another material such as a polymer or metal; OR
v. alloys comprised of 44.5% to 47% titanium, with 0.1 to 2.5% of one or more of the elements including tantalum, iron, molybdenum, manganese, cobalt, chromium, vanadium, niobium, hafnium, zirconium, and tungsten, and the balance nickel; OR
vi. such alloys as described in (v) in combination with (including entwined or side-by-side) another material such as a polymer or metal.
(c) configured at least in part as:
i. a single shape memory wire, tube, filled-tube, or strip
ii. a braided or otherwise twisted shape memory wire, tube, filled-tube, and/or strip

4. A device according to claim 3 where the superelastic ring or ring-like member is superelastic at body temperature, but shape memory at room temperature.

5. A contraceptive device in the form of a female condom with a ring or ring-like member embedded or partially embedded along the perimeter of the polymer material where the ring is
(d) superelastic at room temperature and body temperature,
(e) constructed of a material which is selected from the group of:
i. any alloy or group of alloys which demonstrates the shape memory behavior; OR
ii. such alloy(s) as described in (i) in combination with (including entwined or side-by-side) another material such as a polymer or metal; OR
iii. alloys comprised of 55.4% to 56.1% nickel and 43.9% to 44.6% titanium; OR
iv. such alloys as described in (iii) in combination with (including entwined or side-by-side) another material such as a polymer or metal; OR
v. alloys comprised of 44.5% to 47% titanium, with 0.1 to 2.5% of one or more of the elements including tantalum, iron, molybdenum, manganese, cobalt, chromium, vanadium, niobium, hafnium, zirconium, and tungsten, and the balance nickel; OR
vi. such alloys as described in (v) in combination with (including entwined or side-by-side) another material such as a polymer or metal.
(f) configured at least in part as:
i. a single shape memory wire, tube, filled-tube, or strip
ii. a braided or otherwise twisted shape memory wire, tube, filled-tube, and/or strip

6. A device according to claim 5 where the superelastic ring or ring-like member is superelastic at body temperature, but shape memory at room temperature.
